(19) United States
(12) Patent Application Publication
(10) Pub. No.: US 2026/0055052 A1
(43) Pub. Date: Feb. 26, 2026

(54) PROCESS FOR PREPARING CYANTRANILIPROLE VIA AMINO-CYANO-BENZENE DERIVATIVE

(71) Applicant: ADAMA MAKHTESHIM LTD., Beer Sheva (IL)

(72) Inventors: Gal SUEZ, Dimona (IL); Leonid METSGER, Lehavim (IL); Artur ULIKOWSKI, Olecko (PL); Tomasz JASTRZABEK, Krakow (PL); Rafal LISIAK, Wieliczka (PL); Jie LI, Nanjing (CN)

(73) Assignee: ADAMA MAKHTESHIM LTD., Beer Sheva (IL)

(21) Appl. No.: 19/104,162

(22) PCT Filed: Aug. 13, 2023
(86) PCT No.: PCT/IL2023/050846

§ 371 Date: Feb. 14, 2025

Related U.S. Application Data
(60) Provisional application No. 63/398,247, filed on Aug. 16, 2022.
(60) Provisional application No. 63/456,497, filed on Apr. 2, 2023.

(51) Int. Cl. C07C 249/08; C07C 253/20; C07D 265/26; C07D 401/04

(52) U.S. Cl. CPC C07C 249/08; C07C 253/20; C07D 265/26; C07D 401/04

(57) ABSTRACT

The present invention relates to the preparation of cyantraniliprole, comprising the preparation of 8-methyl-2,4-dioxo-1,4-dihydro-2H-benzo[d][1,3]oxazine-6-carbonitrile key intermediate via 2-amino-5-((hydroxyimino)methyl)-3-methylbenzoic acid. Wherein hydroxylamine attacks a benzylic formyl group, to obtain the 2-amino-5-((hydroxyimino)methyl)-3-methylbenzoic acid, that undergoes simultaneously or by consecutive steps dehydrogenation and cyclization to obtain the benzo[d][1,3]oxazine group and the cyano group of the desired product. In addition, an improved method for the synthesis of the benzylic formyl group is also displayed.

### BACKGROUND

Cyantraniliprole, a compound of formula (VIII)

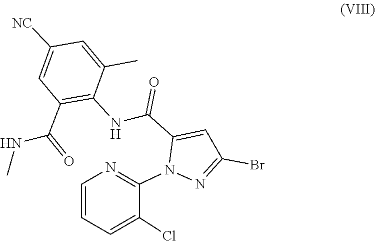 [SMILES: CNC(=O)c1cc(C#N)cc(C)c1NC(=O)c1cc(Br)nn1-c1ncccc1Cl]

is well known for its activity against pesticides i.e., insecticide. This insecticide compound is known for its unique mode of action, the ryanoid class, and thus its highly importance in the agrochemical insecticide industry. E.I. Du Pont De Nemours and Co. were the first to describe Cyantraniliprole, and its family of compounds in WO 2004/067528. In this patent the innovator claims the compound, other corresponding compounds, possible compositions with other insecticides and methods of use. The synthesis of this compound comprises, in some cases, the use and synthesis of one of the key intermediates, 2-amino-5-cyano-3-methylbenzoic acid. The cyano group of this key intermediate is obtained, in most cases, by a coupling reaction of a haloanthranilic acid with a metal cyanide. In some cases, a palladium catalyst or a metal halide is additionally required. This approach is also described in the following patents CN105367548, CN103450154, WO2022058916, CN104003976, WO2006068669, WO2008070158, WO2009111553, WO2009085816, WO2008082502, WO2008070158, WO2009006061. However, this method has many drawbacks such as the use of heavy metals, low efficiency, low yields, high cost and occasionally leads to metal contamination in the final product. Preparative methods for this substance must be improved for economic commercial operation. In view of that, the present invention makes available a convenient, cost-effective route, free of metals way to prepare a 2-amino-5-cyano-3-methylbenzoic acid and its corresponded Cyantraniliprole.

### SUMMARY

The present invention is related to the method of preparing of a compound of formula (I), or its salts thereof

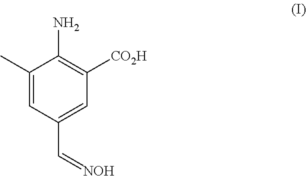

comprising reaction of a compound of formula (II) or its salts thereof

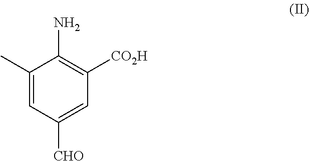

with hydroxylamine, or its salts thereof, optionally in the presence of a solvent, optionally in the presence of a base.

In addition, the present invention is related to the method of preparing of a compound of formula (IV), and its salt thereof,

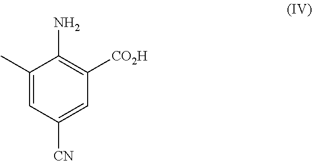

comprising dehydration of a compound of formula (I), optionally in the presence of a solvent.

Another aspect of this present invention is related to the method of preparing of a compound of formula (II), and its salt thereof,

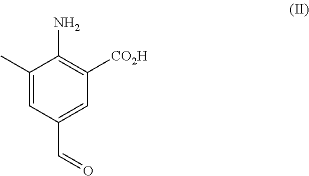

comprising reaction of compound of formula (III), and its salt thereof,

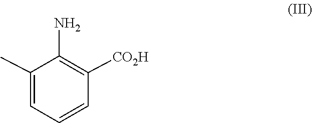

with a formylation reagent selected from hexamethylenetetramine (HMTA), formaldehyde, paraformaldehyde, trioxane and/or methanediol and the mixtures thereof, in the presence of an acid, optionally in the presence of a solvent.

The present invention is also related to the method of preparing of a compound of formula (V), and its salt thereof,

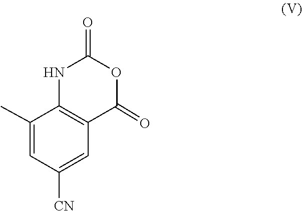

comprising a) reaction of a compound of formula (I) or its salt thereof with a compound of formula (A)

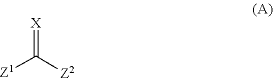

wherein Z¹ and Z², are independently, chloride, C1-C4-alkoxy, trichloromethyloxy, C(O)Cl, C1-C6-alkyloxycarbonate;
X is O;
in the presence of a solvent.

Furthermore, the present invention is related to the method of preparing of a compound of formula (VII), and its salt thereof,

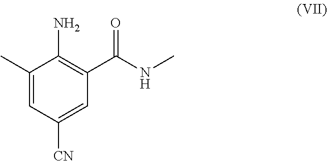

comprising a) reaction of a compound of formula (I) or its salt thereof, prepared by reaction of a compound of formula (II) or its salts thereof

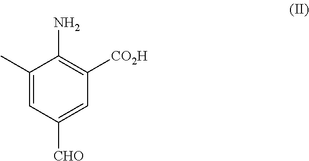

with hydroxylamine, or its salts thereof, optionally in the presence of a solvent, optionally in the presence of a base.
with a compound of formula (A)

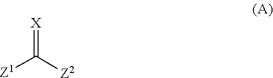

wherein Z¹ and Z², are independently, chloride, C1-C4-alkoxy, trichloromethyloxy, C(O)Cl, C1-C6-alkyloxycarbonate;
X is O;
in the presence of a solvent
and b) further reacting compound of formula (V) with methylamine or its salts thereof, in the presence of a solvent and optionally in the presence of a base.

Additionally, the present invention is related to the method of preparing of a compound of formula (VII), and its salt thereof,

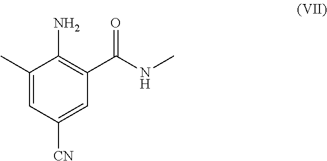

comprising a) reaction of a compound of formula (IV) or its salt thereof, prepared by dehydration of a compound of formula (I), optionally in the presence of a solvent, with a compound of formula (A)

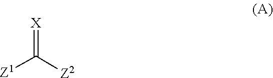

wherein Z¹ and Z², are independently, chloride, C1-C4-alkoxy, trichloromethyloxy, C(O)Cl, C1-C6-alkyloxycarbonate;
X is O;
in the presence of a solvent, optionally in the presence of a base, optionally in the presence of a phase transfer catalyst to obtain a compound of formula (V),

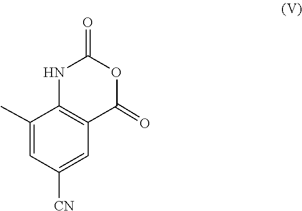

and b) reacting the compound of formula (V) with methylamine or its salts thereof, in the presence of a solvent and optionally in the presence of a base.

Additionally, the present invention is related to the method of preparing of a compound of formula (VII), and its salt thereof,

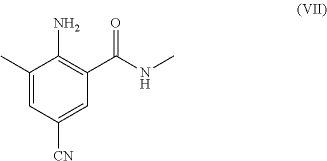

comprising a) reaction of a compound of formula (IV) or its salt thereof, prepared by dehydration of a compound of formula (I), optionally in the presence of a solvent, with thionyl chloride, sulfuryl chloride, phthaloyl chloride, phosphorus pentachloride, phosphorus trichloride, cyanuric chloride, acetic anhydride, propionic anhydride, butyric anhydride, hexanoic anhydride, benzoic anhydride, trichloroacetic anhydride, isopropenyl acetate, acetyl chloride, propionyl chloride, isobutyryl chloride, benzoyl chloride, dicyclohexylcarbodiimide (DCC), diisopropylcarbodiimide (DIC), ethyl-(N′,N′-dimethylamino) propylcarbodiimide hydrochloride (EDC), 1-hydroxybenzotriazole (HOBt), and a mixture thereof optionally in the presence of a base to form a compound of formula (VI)

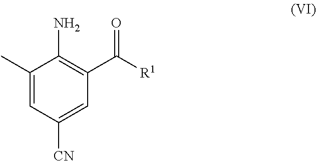

wherein R¹, is halide, optionally halogenated C1-C6-carboxylic acid, carbodiimide, hydroxytriazoles;
and b) reacting the compound of formula (VI) with methylamine or its salts thereof, in the presence of a solvent and optionally in the presence of a base.

The present invention further directed to the process of preparing of a compound of formula (VIII), and its salt thereof,

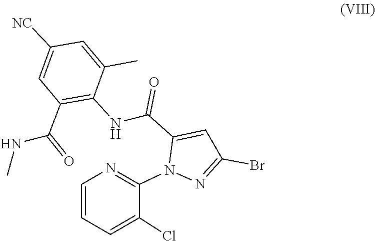 [SMILES: CNC(=O)c1cc(C#N)cc(C)c1NC(=O)c1cc(Br)nn1-c1ncccc1Cl]

using a compound of formula (I) or its salt thereof.

The present invention is also directed to the process of preparing of a compound of formula (VIII), and its salt thereof,

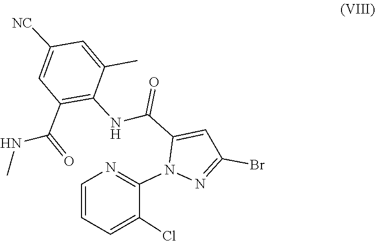 [SMILES: CNC(=O)c1cc(C#N)cc(C)c1NC(=O)c1cc(Br)nn1-c1ncccc1Cl]

using a compound of formula (IV) or its salt thereof, prepared by dehydration of a compound of formula (I), optionally in the presence of a solvent.

### DESCRIPTION OF THE INVENTION

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

Throughout the application, descriptions of various embodiments use the term “comprising”; however, it will be understood by one skilled in the art, that in some specific instances, an embodiment can alternatively be described using the language “consisting essentially of” or “consisting of”.

The term “a” or “an” as used herein includes the singular and the plural, unless specifically stated otherwise. Therefore, the terms “a,” “an” or “at least one” can be used interchangeably in this application.

The term “alkyl” as used herein refers to a branched, unbranched, or cyclic carbon chain.

The term “halogen” or “halo” as used herein refers to one or more halogen atoms, defined as F, Cl, Br, and I. Unless indicated to the contrary, the numerical parameters set forth in the following specification and attached claims are approximations that may vary depending upon the desired properties sought to be obtained. For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.”

The term “carbonyl” as used herein refers to the group —C═O.

The term “alkoxy,” as used herein, refers to an alkyl group attached to the parent molecular moiety through an oxygen atom.

The term “alkoxycarbonyl,” as used herein, refers to an alkoxy group attached to the parent molecular moiety through a carbonyl group.

The term “trichloromethyloxy,” as used herein, refers to a trichloromethyl group attached to the parent molecular moiety through an oxygen atom.

The term “alkyloxycarbonate” as used herein, refers to an alkoxy group attached to the parent molecular moiety through a carbonate group.

The term “telescopic process” as used herein refers to carrying out several reactions without isolating the intermediate products. In particular, the telescopic process suggests the execution of multiple transformations (including reaction quenches and other workup operations) without the direct isolation of intermediates. Telescoped solutions of intermediates can be extracted, filtered (as long as the desired product remains in the filtrate), and solvent exchanged, but the intermediate is ultimately held in solution and carried forward to the subsequent transformation.

The term “salts”, as used herein, refers to organic salts such as chloride, bromide, fluoride, iodide, acetate, hydrogen sulfates, phosphates, formats, nitrate, carbonate etc., or, if applicable, alkaline metal salts such as sodium, potassium, calcium, lithium, cesium, magnesium, barium and the like.

Any of the compounds described here as basic compound or intermediate in a process is intended also to include the compound salts such as HCl salts, acetic acid salts etc., no special meaning should be given to the fact that in some cases this is mentioned or not mention for specific compound in the text.

The salts of the compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

At the very least, each numerical parameter should at least be construed in light of the number of reported significant digits and by applying ordinary rounding techniques. In an embodiment, use of the term “about” herein specifically includes ±10% from the indicated values in the range. In addition, the endpoints of all ranges directed to the same component or property herein are inclusive of the endpoints, are independently combinable, and include all intermediate points and ranges.

In an embodiment, the present invention provides a method for preparing of a compound of formula (I), or its salts thereof

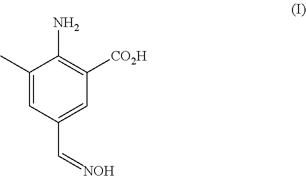

comprising reaction of a compound of formula (II) or its salts thereof

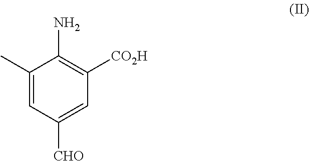

with hydroxylamine, or its salts thereof, optionally in the presence of a solvent, optionally in the presence of a base.

According to an embodiment, the hydroxylamine salt is selected from a group comprising hydroxylamine hydrochloride, hydroxylamine hydrobromide, hydroxylamine acetate, hydroxylamine fluoride, hydroxylamine iodide, hydroxylamine sulfate, hydroxylamine disulfate, hydroxylamine phosphate, hydroxylamine nitrate, Hydroxylamine perchlorate, Hydroxylamine-O-sulfonic acid, hydroxylamine carbonate and the mixture thereof.

According to an embodiment, the hydroxylamine salt is selected from a group comprising hydroxylamine hydrochloride, hydroxylamine acetate, hydroxylamine sulfate, hydroxylamine phosphate, hydroxylamine nitrate, and the mixture thereof.

According to an embodiment, the hydroxylamine salt is hydroxylamine hydrochloride.

According to an embodiment, a molar ratio between the compound of formula (II) to hydroxylamine or hydroxylamine salt can be from about 1:10 to 10:1, preferably from about 1:5 to 1:1, most preferably from about 1:1.2 to 1:1.

According to an embodiment, a molar ratio between the compound of formula (II) to hydroxylamine or hydroxylamine salt can be from about 1:5 to 1:0.9, preferably from about 1:3 to 1:1, most preferably from about 1:1.15 to 1:1.05.

According to an embodiment the base is selected from a group comprising triethylamine, dimethylamine, aniline, indole, piperidine, pyridine, pyrimidine, pyrrolidine, pyrrole, imidazole, methylimidazole, 2-picoline, 4-methylmorpholine, dimethylaminopyridine, N,N-diisopropylethylamine, sodium carbonate, sodium bicarbonate, sodium hydroxide, sodium acetate, potassium carbonate, potassium bicarbonate, potassium hydroxide, aluminum hydroxide, calcium hydroxide, iron hydroxide, lithium hydroxide, ammonium hydroxide, ammonium acetate, and a mixture thereof.

According to an embodiment, a molar ratio between the compound of formula (II) to the base can be from about 1:10 to 10:1, preferably from about 1:5 to 1:1, most preferably from about 1:1.2 to 1:1.

According to an embodiment, a molar ratio between the compound of formula (II) to the base can be from about 1:5 to 1:0.9, preferably from about 1:3 to 1:1, most preferably from about 1:1.2 to 1:1.05.

According to an embodiment the solvent is selected from a group comprising aliphatic cyclic and acyclic hydrocarbons, such as octane, heptane, hexane, pentane, cyclooctane, cyclohexane, cyclopentane petroleum ether, halogenated aliphatic cyclic and acyclic hydrocarbons such as, carbon tetrachloride, chloroform, methylenechloride, 1,2-dichloroethane, aromatic hydrocarbons such as benzene, toluene, xylene, ethylbenzene, halogenated aromatic hydrocarbons such as chlorobenzene, dichlorobenzene, trichlorobenzene, aliphatic cyclic ethers such as diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), cyclopentylmethyl ether, aliphatic and cyclic esters such as ethyl acetate, nitriles such as acetonitrile, benzonitrile, ketones such as acetone, 2-butanone, C1-C6 alcohols such as methanol, ethanol, 1-butanol, 2-butanol, 1-propanol, 2-propanol, t-butyl alcohol, diethylene glycol, glycerin, ethylene glycol, propylene glycol, C1-C4 carboxylic acids such as acetic acid, propionic acid, benzylic acid, polar protic and aprotic solvents such as formic acid, N-methyl-2-pyrrolidone, dimethylformamide, dimethylacetamide, dimethylsulfoxide, water, and a mixture thereof.

According to an embodiment the solvent is selected from a group comprising, halogenated aliphatic cyclic and acyclic hydrocarbons such as chloroform, methylenechloride, aliphatic ethers such as diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), aliphatic esters such as ethyl acetate, nitriles such as acetonitrile, ketones such as acetone, 2-butanone, polar protic and aprotic solvents such as formic acid, N-methyl-2-pyrrolidone, dimethylformamide, dimethylacetamide, dimethylsulfoxide, water, and a mixture thereof.

According to an embodiment, a w/w ratio between the compound of formula (II) to organic solvent can be from about 1:20 to 10:1, preferably from about 1:5 to 1:0.5, most preferably from about 1:5 to 1:3.

According to an embodiment, a w/w ratio between the compound of formula (II) to organic solvent can be from about 1:10 to 1:1, preferably from about 1:5 to 1:1, most preferably from about 1:5 to 1:4.

According to an embodiment, the compound of formula (II) contacted with the hydroxylamine or its salt thereof at the temperature interval of from about 10 to 130° C. A preferred temperature interval is from about 50 to 100° C., more preferably from about 80 to 90° C.

According to an embodiment, the compound of formula (II) contacted with the hydroxylamine or its salt thereof at the temperature interval of from about 50 to 100° C. A preferred temperature interval is from about 70 to 90° C., more preferably from about 80 to 85° C.

According to an embodiment the reaction mixture is monitored by HPLC analytical method, and the process ends when concentration of formula (II) is between 0-99%, preferably from 0-50%. In particular, the process should be terminated when no more than 5% of compound of formula (II) remains in the reaction media.

According to an embodiment the reaction mixture is monitored by HPLC analytical method, and the process ends when concentration of formula (II) is between 0-40%, preferably from 0-10%.

In particular, the process should be terminated when no more than 1% of compound of formula (II) remains in the reaction media.

Finally, the reaction mixture containing the resulting a compound of formula (I) is optionally worked up or proceed as in telescopic/one-pot reaction without workup to the next step of dehydration. This stage may include adding water, adding organic solvent, stirring, cooling, heating, phases separation, distillation, precipitation, recrystallization, concentration, filtration, purification, pH adjustment, extraction, and drying processes.

In additional embodiment, the present invention provides a method of preparing of a compound of formula (IV), and its salt thereof,

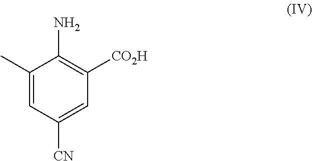

comprising dehydration of a compound of formula (I), optionally in the presence of a dehydration agent, optionally in the presence of a solvent.

According to embodiments the processes of preparing of the compound of formula (IV) comprises two approaches, wherein the first approach is a straightforward synthesis of compound of formula (IV) from compound of formula (I) and the second approach is the synthesis of compound of formula (IV) from compound of formula (II) via the formation of compound of formula (I), which can be completed as separated processes or as combined processes, such as one-pot reaction, telescopic-reaction, preferably, in one-pot reaction process. The intermediates formed in the above process can be isolated from the reaction mixture or the process can be continued without isolation of said intermediates.

According to an embodiment the dehydration of a compound of formula (I), could be performed in the present of dehydration agent selected from a group comprising p-toluenesulfonic acid, methanesulfonic acid, trifluoromethanesulfonic acid, trifluoroacetic acid, trichloroacetic acid, trimethylsilyl triflate, calcium chloride, iron chloride, aluminum oxide, silicon dioxide, hydrochloric acid, sulfuric acid, phosphoric acid, methanesulfonyl chloride, acetic acid, oxalic acid, dimethyl sulfoxide and the mixtures thereof.

According to an embodiment the dehydration of a compound of formula (I), could be performed in the presence of dehydration agent selected from a group comprising p-toluenesulfonic acid, methanesulfonic acid, sulfuric acid, calcium chloride dimethyl sulfoxide and the mixtures thereof.

According to an embodiment, a molar ratio between the compound of formula (I) to dehydration agent can be from about 1:20 to 10:1, preferably from about 1:1 to 1:5, most preferably from about 1:3 to 1:5.

According to an embodiment, a molar ratio between the compound of formula (I) to dehydration agent can be from about 1:10 to 5:1, preferably from about 1:2 to 1:5, most preferably from about 1:4 to 1:5.

According to an embodiment the solvent is selected from a group comprising aliphatic cyclic and acyclic hydrocarbons, such as octane, heptane, hexane, pentane, cyclooctane, cyclohexane, cyclopentane petroleum ether, halogenated aliphatic cyclic and acyclic hydrocarbons such as, carbontetrachloride, chloroform, methylenechloride, 1,2-dichloroethane, aromatic hydrocarbons such as benzene, toluene, xylene, ethylbenzene, halogenated aromatic hydrocarbons such as chlorobenzene, dichlorobenzene, trichlorobenzene, aliphatic and cyclic ethers such as ether, diethyl glycol diglyme (diethylene dimethyl ether), 1,4-dioxane, methyl t-butyl ether (MTBE), isopropylmethyl ether, tetrahydrofuran (THF), methyl-tetrahydrofuran (Me-THF), cyclopentylmethyl ether, aliphatic and cyclic esters such as ethyl acetate nitriles such as acetonitrile, benzonitrile, ketones such as acetone, 2-butanone, C1-C6 alcohols such as methanol, ethanol, 1-butanol, 2-butanol, 1-propanol, 2-propanol, t-butyl alcohol, diethylene glycol, glycerin, ethylene glycol, propylene glycol polar protic and aprotic solvents N-methyl-2-pyrrolidone, dimethylformamide, dimethylacetamide, dimethylsulfoxide, water, and a mixture thereof.

According to an embodiment the solvent is selected from a group comprising, halogenated aliphatic cyclic and acyclic hydrocarbons such as chloroform, methylenechloride, aliphatic ethers such as diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), aliphatic esters such as ethyl acetate, nitriles such as acetonitrile, ketones such as acetone, 2-butanone, polar protic and aprotic solvents such as formic acid, N-methyl-2-pyrrolidone, dimethylformamide, dimethylacetamide, dimethylsulfoxide, water, and a mixture thereof.

According to an embodiment, a w/w ratio between the compound of formula (I) to organic solvent can be from about 1:20 to 10:1, preferably from about 1:5 to 1:0.5, most preferably from about 1:5 to 1:3.

According to an embodiment, a w/w ratio between the compound of formula (I) to organic solvent can be from about 1:10 to 1:1, preferably from 1:5 to 1:1, most preferably from about 1:5 to 1:4.

According to an embodiment, the dehydration process of compound of formula (I) can be performed at the temperature interval of from about 10 to 130° C. A preferred temperature interval is from about 50 to 100° C., more preferably from about 80 to 90° C.

According to an embodiment, the dehydration process compound of formula (I) can be performed at the temperature interval of from about 50 to 110° C. A preferred temperature interval is from about 70 to 90° C., more preferably from about 85 to 90° C.

According to an embodiment the reaction mixture is monitored by HPLC analytical method, and the process ends when concentration of formula (I) is between 0-99%, preferably from 0-50%. In particular, the process should be terminated when no more than 5% of compound of formula (I) remains in the reaction media.

According to an embodiment the reaction mixture is monitored by HPLC analytical method, and the process ends when concentration of formula (I) is between 0-40%, preferably from 0-10%. In particular, the process should be terminated when no more than 1% of compound of formula (I) remains in the reaction media.

Finally, the reaction mixture containing the resulting a compound of formula (IV) is worked up. This stage may include adding water, adding organic solvent, stirring, cooling, heating, phases separation, distillation, precipitation, recrystallization, concentration, filtration, purification, pH adjustment, extraction, and drying processes.

In another embodiment, the present invention provides a method of preparing of a compound of formula (II), and its salt thereof,

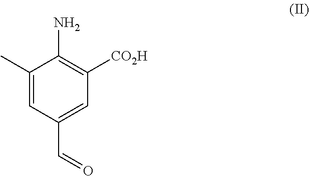

comprising reaction of compound of formula (III), and its salt thereof,

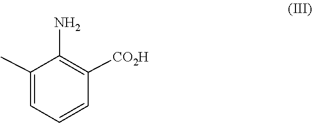

with a formylation reagent selected from hexamethylenetetramine (HMTA), formaldehyde, paraformaldehyde, trioxane and/or methanediol and the mixtures thereof, in the presence of an acid, optionally in the presence of a solvent.

The compound of formula (III) (3-methyl-anthranilic acid, CAS number [4389-45-1]) is commercially available and known compound which could be prepared according to numerous common methods.

According to an embodiment the molar ratio between the compound of formula (III) to the formylation reagent, can be from about 1:20 to 1:0.3, preferably from about 1:5 to 1:0.5, most preferably from about 1:2 to 1:1.

According to an embodiment the molar ratio between the compound of formula (III) to the formylation reagent, can be from about 1:5 to 1:0.3, preferably from about 1:5 to 1:1, most preferably from about 1:1.2 to 1:1.1.

According to an embodiment the molar ratio between the compound of formula (III) to the formylation reagent can be from about 1:20 to 1:0.1, preferably from about 1:5 to 1:0.5, most preferably from about 1:0.8 to 1:0.6.

According to an embodiment the molar ratio between the compound of formula (III) to the formylation reagent can be from about 1:10 to 1:0.5, preferably from about 1:2 to 1:0.5, most preferably from about 1:0.7 to 1:0.5.

According to an embodiment the formylation reagent is selected form the group comprising hexamethylenetetramine (HMTA), formaldehyde, paraformaldehyde, trioxane, methanediol, and the mixtures thereof.

According to an embodiment the formylation reagent is hexamethylenetetramine (HMTA).

According to an embodiment the formylation reagent is formaldehyde.

According to an embodiment the formylation reagent is paraformaldehyde.

According to an embodiment the acid is selected from a group comprising, formic acid, triflic acid, oxalic acid, carbonic acid, citric acid, tartartic acid, glutaric acid, lactic acid, malonic acid, monochloroacetic acid, dichloroacetic acid, trichloroacetic acid, trifluoroacetic acid, sulfuric acid, phosphoric acid, nitric acid, methanesulfonic acid, para-toluenesulfonic acid, hydrochloric acid, hydrobromic acid, boric acid and the mixture thereof.

According to an embodiment the acid is selected from a group comprising methanesulfonic acid, para-toluenesulfonic acid, sulfuric acid, and the mixture thereof.

According to an embodiment, a molar ratio between the compound of formula (III) to the acid can be from about 1:20 to 1:0.1, preferably from about 1:10 to 1:0.5, most preferably from about 1:5 to 1:1.

According to an embodiment, a molar ratio between the compound of formula (III) to the acid can be from about 1:10 to 1:1, preferably from about 1:3 to 1:1, most preferably from about 1:1.9 to 1:1.5.

According to an embodiment the solvent is selected from a group comprising aliphatic cyclic and acyclic hydrocarbons, such as octane, heptane, hexane, pentane, cyclooctane, cyclohexane, cyclopentane petroleum ether, halogenated aliphatic cyclic and acyclic hydrocarbons such as, methylenechloride, 1,2-dichloroethane, aromatic carbontetrachloride, chloroform, hydrocarbons such as benzene, toluene, xylene, ethylbenzene, halogenated aromatic hydrocarbons such as chlorobenzene, dichlorobenzene, trichlorobenzene, aliphatic and cyclic ethers diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), cyclopentylmethyl ether, aliphatic and cyclic esters such as ethyl acetate, nitriles such as acetonitrile, benzonitrile, ketones such as acetone, 2-butanone, C1-C6 alcohols such as methanol, ethanol, 1-butanol, 2-butanol, 1-propanol, 2-propanol, t-butyl alcohol, diethylene glycol, glycerin, ethylene glycol, propylene glycol polar protic such as and aprotic solvents N-methyl-2-pyrrolidone, dimethylformamide, dimethylacetamide, dimethylsulfoxide, acetic acid, water, and a mixture thereof.

According to an embodiment the solvent is selected from a group comprising halogenated aliphatic acyclic hydrocarbons such as, methylenechloride, halogenated aromatic hydrocarbons such as chlorobenzene, dichlorobenzene, trichlorobenzene, aliphatic and cyclic ethers diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), aliphatic esters such as ethyl acetate, nitriles such as acetonitrile, ketones such as acetone, 2-butanone, C1-C6 alcohols such as methanol, ethanol, 1-butanol, 1-propanol, 2-propanol, polar protic and aprotic solvents such as N-methyl-2-pyrrolidone, dimethylformamide, dimethylacetamide, dimethylsulfoxide, acetic acid, water, and a mixture thereof.

According to an embodiment the solvent is selected from a group comprising chlorobenzene, N-methyl-2-pyrrolidone, dimethylformamide, dimethylacetamide, dimethylsulfoxide, acetic acid water, and a mixture thereof.

According to an embodiment, a w/w ratio between the compound of formula (III) to organic solvent can be from about 1:20 to 10:1, preferably from about 1:5 to 1:1, most preferably from about 1:3 to 1:1.

According to an embodiment, a w/w ratio between the compound of formula (III) to organic solvent can be from about 1:10 to 1:1, preferably from about 1:5 to 1:1, most preferably from about 1:2.2 to 1:2.

According to an embodiment, compound of formula (III) contacted with the formylation reagent is performed between the temperature interval of from about 10 to 130° C. A preferred temperature interval is from about 70 to 120° C.

According to an embodiment, compound of formula (III) contacted with the formylation reagent is performed between the temperature interval of from about 70 to 120° C. A preferred temperature interval is from about 80 to 100° C., more preferably from about 85 to 95° C.

According to an embodiment the reaction mixture is monitored by HPLC analytical method, and the process ends when concentration of formula (III) is between 0-99%, preferably from 0-50%. In particular, the process should be terminated when no more than 1% of compound of formula (III) remains in the reaction media.

According to an embodiment the reaction mixture is monitored by HPLC analytical method, and the process ends when concentration of formula (III) is between 0-40%, preferably from 0-10%. In particular, the process should be terminated when no more than 1% of compound of formula (III) remains in the reaction media.

Finally, the reaction mixture containing the resulting a compound of formula (II) is worked up. This stage may include adding water, adding organic solvent, stirring, cooling, heating, phases separation, distillation, precipitation, recrystallization, concentration, filtration, purification, pH adjustment, extraction, and drying processes.

In another embodiment, the present invention provides a method of preparing of a compound of formula (V), and its salt thereof,

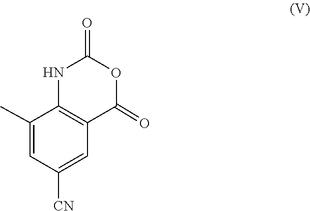

comprising a) reaction of a compound of formula (I) or its salt thereof, prepared by reaction of a compound of formula (II) or its salts thereof

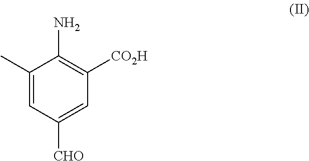

with hydroxylamine, or its salts thereof, optionally in the presence of a solvent, optionally in the presence of a base.
with a compound of formula (A)

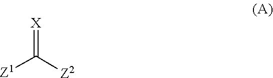

wherein Z¹ and Z², are independently, chloride, C1-C4-alkoxy, trichloromethyloxy, C(O)Cl, C1-C6-alkyloxycarbonate;
X is O;
in the presence of a solvent.

According to an embodiment the solvent is selected from a group comprising aliphatic cyclic and acyclic hydrocarbons, such as octane, heptane, hexane, pentane, cyclooctane, cyclohexane, cyclopentane petroleum ether, halogenated aliphatic cyclic and acyclic hydrocarbons such as, carbontetrachloride, chloroform, methylenechloride, 1,2-dichloroethane, aromatic hydrocarbons such as benzene, toluene, xylene, ethylbenzene, halogenated aromatic hydrocarbons such as chlorobenzene, dichlorobenzene, trichlorobenzene, aliphatic and cyclic ethers diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), cyclopentylmethyl ether, aliphatic and cyclic esters such as ethyl acetate, nitriles such as acetonitrile, benzonitrile, ketones such as acetone, 2-butanone, C1-C6 alcohols such as methanol, ethanol, 1-butanol, 2-butanol, 1-propanol, 2-propanol, t-butyl alcohol, diethylene glycol, glycerin, ethylene glycol, propylene glycol polar protic such as and aprotic solvents N-methyl-2-pyrrolidone, dimethylformamide, dimethylacetamide, dimethylsulfoxide, acetic acid, water, and a mixture thereof.

According to an embodiment the solvent is selected from a group comprising halogenated aliphatic acyclic hydrocarbons such as, methylenechloride, halogenated aromatic hydrocarbons such as chlorobenzene, dichlorobenzene, trichlorobenzene, aliphatic and cyclic ethers diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), aliphatic esters such as ethyl acetate, nitriles such as acetonitrile, ketones such as acetone, 2-butanone, C1-C6 alcohols such as methanol, ethanol, 1-butanol, 1-propanol, 2-propanol, polar protic and aprotic solvents such as N-methyl-2-pyrrolidone, dimethylformamide, dimethylacetamide, dimethylsulfoxide, acetic acid, water, and a mixture thereof.

According to an embodiment the solvent is selected from a group comprising aliphatic and cyclic ethers diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), and a mixture thereof.

According to an embodiment, a w/w ratio between the compound of formula (I) to the solvent can be from about 1:20 to 10:1, preferably from about 1:1 to 1:10, most preferably from about 1:3 to 1:5.

According to an embodiment, a w/w ratio between the compound of formula (I) to the solvent in step a) can be from about 1:1 to 1:10, preferably from about 1:1 to 1:5, most preferably from about 1:3 to 1:4.

According to an embodiment the compound of formula (A) is selected form a group comprises of phosgene, diphosgene, triphosgene, methyl chloroformate, ethyl chloroformate, dimethylcarbamoyl chloride, oxalyl chloride, di-t-butyl dicarbonate, dimethyl dicarbonate, diethyl dicarbonate or a mixture thereof.

According to an embodiment the compound of formula (A) is selected form a group comprises of triphosgene, phosgene, oxalyl chloride, or a mixture thereof.

According to an embodiment, a w/w ratio between the compound of formula (I) to compound of formula (A) can be from about 1:20 to 10:1, preferably from about 1:1 to 1:10, most preferably from about 1:1 to 1:2.

According to an embodiment, a w/w ratio between the compound of formula (I) to compound of formula (A) in step a) can be from about 1:1 to 1:10, preferably from about 1:1 to 1:5, most preferably from about 1:1.05 to 1:1.2.

According to an embodiment, compound of formula (I) contacted with compound of formula (A) is performed between the temperature interval of from about 0 to 100° C. A preferred temperature interval is from about 0 to 60° C., more preferably from about 20 to 40° C.

According to an embodiment, compound of formula (I) contacted with compound of formula (A) is performed between the temperature interval of from about 0 to 60° C. A preferred temperature interval is from about 20 to 40° C., more preferably from about 25 to 30° C.

According to an embodiment the reaction mixture is monitored by HPLC analytical method, and the process ends when concentration of formula (I) is between 0-99%, preferably from 0-50%, most preferably, in particular, when no more than 1% of compound of formula (I) remains in the reaction media.

According to an embodiment the reaction mixture is monitored by HPLC analytical method, and the process ends when concentration of formula (I) is between 0-40%, preferably from 0-10%. In particular the process ends when no more than 1% of compound of formula (I) remains in the reaction media.

Finally, the reaction mixture containing the resulting a compound of formula (I) is worked up. This stage may include adding water, adding organic solvent, stirring, cooling, heating, phases separation, distillation, precipitation, recrystallization, concentration, filtration, purification, pH adjustment, extraction, and drying processes.

In another embodiment, the present invention provides a method of preparing of a compound of formula (VII), and its salt thereof,

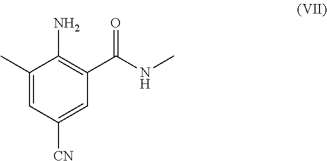

comprising a) reaction of a compound of formula (IV) or its salt thereof, prepared according to the present invention, with a compound of formula (A)

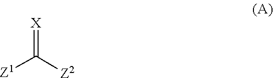

wherein Z¹ and Z², are independently, chloride, C1-C4-alkoxy, trichloromethyloxy, C(O)Cl, C1-C6-alkyloxycarbonate;
X is O;
in the presence of a solvent, optionally in the presence of a base, optionally in the presence of a phase transfer catalyst to obtain a compound of formula (V),

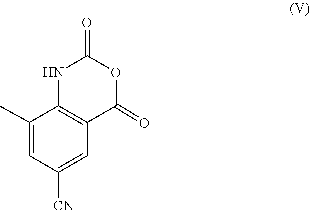

and b) reacting the compound of formula (V) with methylamine or its salts thereof, in the presence of a solvent and optionally in the presence of a base.

According to an embodiment the compound of formula (A) is selected form a group comprises of phosgene, diphosgene, triphosgene, methyl chloroformate, ethyl chloroformate, dimethylcarbamoyl chloride, oxalyl chloride, di-t-butyl dicarbonate, dimethyl dicarbonate, diethyl dicarbonate or a mixture thereof.

According to an embodiment the compound of formula (A) is selected form a group comprises of triphosgene, phosgene, oxalyl chloride, or a mixture thereof.

According to an embodiment, a molar ratio between the compound of formula (IV) to the compound of formula (A) is about 1:20 to 1:0.1, preferably from about 1:5 to 1:0.2, most preferably from about 1:1 to 1:0.4.

According to an embodiment, a molar ratio between the compound of formula (IV) to the compound of formula (A) is about 1:10 to 1:0.1, preferably from about 1:1 to 1:0.2, most preferably from about 1:0.6 to 1:0.4.

According to an embodiment the base is selected from a group comprising triethylamine, dimethylamine, aniline, indole, piperidine, pyridine, pyrimidine, pyrrolidine, pyrrole, imidazole, methylimidazole, 2-picoline, 4-methylmorpholine, dimethylaminopyridine, N,N-diisopropylethylamine, sodium carbonate, sodium bicarbonate, sodium hydroxide, sodium acetate, potassium carbonate, potassium bicarbonate, potassium hydroxide, aluminum hydroxide, calcium hydroxide, iron hydroxide, lithium hydroxide, ammonium hydroxide, ammonium acetate, and a mixture thereof.

According to an embodiment the base is selected from a group comprising triethylamine, imidazole, sodium hydroxide, potassium hydroxide, and a mixture thereof.

According to an embodiment, a molar ratio between the compound of formula (IV) to the base can be from about 1:20 to 1:1, preferably from about 1:10 to 1:1.2, most preferably from about 1:5 to 1:2.

According to an embodiment, a molar ratio between the compound of formula (IV) to the base can be from about 1:10 to 1:1, preferably from about 1:5 to 1:1.2, most preferably from about 1:3 to 1:2.

According to an embodiment the phase transfer catalyst selected from the group consisting of ammonium salts or polyethers selected from the group consisting of pyridinium hydrochloride, pyridinium acetate, pyridinium triflate, pyridinium hydrobromide, tetrabutylammonium bromide, tetrabutylammonium chloride, tetrabutylammonium fluoride, tetrabutylammonium hydrogen sulfate, tetrabutylammonium iodide, crown ethers, polyethylene glycol, and the mixtures thereof.

According to an embodiment the phase transfer catalyst selected from the group consisting of ammonium salts or polyethers selected from the group consisting of tetrabutylammonium bromide, tetrabutylammonium chloride, polyethylene glycol, and the mixtures thereof.

According to an embodiment, a molar ratio between the compound of formula (IV) to the phase transfer catalyst can be from about 1:1 to 1:0.0001, preferably from about 1:0.1 to 1:0.001, most preferably from about 1:0.05 to 1:0.005.

According to an embodiment, a molar ratio between the compound of formula (IV) to the phase transfer catalyst can be from about 1:0.1 to 1:0.001, preferably from about 1:0.05 to 1:0.01, most preferably from about 1:0.04 to 1:0.02.

According to an embodiment the solvent in step a) is selected from a group comprising aliphatic cyclic and acyclic hydrocarbons such as octane, heptane, hexane, pentane, cyclooctane, cyclohexane, cyclopentane petroleum ether, halogenated aliphatic cyclic and acyclic hydrocarbons such as carbontetrachloride, chloroform, methylenechloride, 1,2-dichloroethane, aromatic hydrocarbons benzene, toluene, xylene, ethylbenzene, halogenated aromatic hydrocarbons such as chlorobenzene, dichlorobenzene, trichlorobenzene, aliphatic and cyclic ethers such as diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), cyclopentylmethyl ether, aliphatic and esters such as ethyl acetate, nitriles such as acetonitrile, benzonitrile, ketones such as acetone, 2-butanone, dimethyl carbonate, polar protic and aprotic solvents such as dimethylformamide, pyridine, dimethylsulfoxide, n-alkylpyrrolidones, and a mixture thereof.

According to an embodiment the solvent in step a) is selected from a group comprising halogenated aliphatic acyclic hydrocarbons such as methylenechloride, aromatic hydrocarbons such as toluene, halogenated aromatic hydrocarbons such as chlorobenzene, dichlorobenzene, trichlorobenzene, aliphatic and cyclic ethers such as 1,4-dioxane, methyl t-butyl ether (MTBE), isopropylmethyl ether, tetrahydrofuran (THF), methyl-tetrahydrofuran (Me-THF), aliphatic esters such as ethyl acetate, nitriles such as acetonitrile, polar protic and aprotic solvents such as dimethylformamide, pyridine, dimethylsulfoxide, n-alkylpyrrolidones, and a mixture thereof.

According to an embodiment, a w/w ratio between the compound of formula (IV) to the solvent in step a) can be from about 1:20 to 10:1, preferably from about 1:0.5 to 1:5, most preferably from about 1:3 to 1:5.

According to an embodiment, a w/w ratio between the compound of formula (IV) to the solvent in step a) can be from about 1:10 to 1:1, preferably from about 1:5 to 1:1, most preferably from about 1:3 to 1:4.

According to an embodiment, compound of formula (IV) contacted with compound of formula (A) is performed between the temperature interval of from about 0 to 100° C. A preferred temperature interval is from about 10 to 60° C., more preferably from about 20 to 30° C.

According to an embodiment, compound of formula (IV) contacted with compound of formula (A) is performed between the temperature interval of from about 5 to 60° C. A preferred temperature interval is from about 10 to 40° C., more preferably from about 20 to 30° C.

According to an embodiment, compound of formula (IV) contacted with triphosgene is performed between the temperature interval of from about 5 to 60° C. A preferred temperature interval is from about 10 to 40° C., more preferably from about 20 to 30° C.

According to an embodiment the reaction mixture is monitored by HPLC analytical method, and the process ends when concentration of formula (IV) is between 0-99%, preferably from 0-50%, most preferably, in particular, when no more than 1% of compound of formula (IV) remains in the reaction media.

According to an embodiment the reaction mixture is monitored by HPLC analytical method, and the process ends when concentration of formula (IV) is between 0-40%, preferably from 0-10%.

In particular the process ends when no more than 1% of compound of formula (IV) remains in the reaction media.

Finally, the reaction mixture containing the resulting a compound of formula (IV) is worked up. This stage may include adding water, adding organic solvent, stirring, cooling, heating, phases separation, distillation, precipitation, recrystallization, concentration, filtration, purification, pH adjustment, extraction, and drying processes.

According to an embodiment methylamine salt is selected from a group comprising of methylammonium chloride, methylammonium bromide, methylammonium iodide, methylammonium nitrate, methylammonium format, methylammonium sulfate, methylammonium tetrafluoroborate, methylammonium acetate, methylammonium hydroxide, methylammonium perchlorate and a mixture thereof.

According to an embodiment methylamine salt is selected from a group comprising of methylammonium chloride, methylammonium nitrate, methylammonium acetate, methylammonium hydroxide and a mixture thereof.

According to an embodiment, a molar ratio between compound (V) to the methylamine or its salt thereof can be from about 1:20 to 1:0.1, preferably from about 1:10 to 1:1, most preferably from about 1:5 to 1:3.

According to an embodiment, a molar ratio between compound (V) to the methylamine or its salt thereof can be from about 1:10 to 1:1, preferably from about 1:5 to 1:2, most preferably from about 1:3 to 1:2.

According to an embodiment the solvent in step b) is selected from a group comprising aliphatic cyclic and acyclic hydrocarbons, such as octane, heptane, hexane, pentane, cyclooctane, cyclohexane, cyclopentane petroleum ether, halogenated aliphatic cyclic and acyclic hydrocarbons such as, carbontetrachloride, chloroform, methylenechloride, 1,2-dichloroethane, aromatic hydrocarbons such as benzene, toluene, xylene, ethylbenzene, halogenated aromatic hydrocarbons such as chlorobenzene, dichlorobenzene, trichlorobenzene, aliphatic and cyclic ethers such as diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), cyclopentylmethyl ether, aliphatic esters such as ethyl acetate nitriles such as acetonitrile, benzonitrile, ketones such as acetone, 2-butanone, C1-C6 alcohols such as methanol, ethanol, 1-butanol, 2-butanol, 1-propanol, 2-propanol, t-butyl alcohol, diethylene glycol, glycerin, ethylene glycol, propylene glycol polar protic and aprotic solvents N-methyl-2-pyrrolidone, dimethylformamide, dimethylacetamide, dimethylsulfoxide, water, and a mixture thereof.

According to an embodiment the solvent in step b) is selected from a group comprising halogenated aliphatic hydrocarbons such as, methylenechloride, aliphatic and cyclic ethers such 1,4-dioxane, methyl t-butyl ether (MTBE), isopropylmethyl ether, tetrahydrofuran (THF), methyl-tetrahydrofuran (Me-THF), aliphatic esters such as ethyl acetate, nitriles such as acetonitrile, C1-C6 alcohols such as methanol, ethanol, 1-butanol, 2-propanol, polar protic and aprotic solvents such as N-methyl-2-pyrrolidone, dimethylformamide, dimethylacetamide, dimethylsulfoxide, water, and a mixture thereof.

According to an embodiment, a w/w ratio between the compound of formula (V) to the solvent in step a) can be from about 1:20 to 10:1, preferably from about 1:0.5 to 1:5, most preferably from about 1:3 to 1:5.

According to an embodiment, a w/w ratio between the compound of formula (V) to the solvent in step a) can be from about 1:10 to 1:1, preferably from about 1:5 to 1:1, most preferably from about 1:3 to 1:4.

According to an embodiment, compound of formula (V) contacted with methylamine, or its salt thereof is performed between the temperature interval of from about-10 to 100° C. A preferred temperature interval is from about 5 to 30° C., more preferably from about 20 to 30° C.

According to an embodiment, compound of formula (V) contacted with methylamine, or its salt thereof is performed between the temperature interval of from about 0 to 50° C. A preferred temperature interval is from about 10 to 40° C., more preferably from about 25 to 30° C.

According to an embodiment the reaction mixture is monitored by HPLC analytical method, and the process ends when concentration of formula (V) is between 0-99%, preferably from 0-50%. In particular, the process should be terminated when no more than 1% of compound of formula (V) remains in the reaction media.

According to an embodiment the reaction mixture is monitored by HPLC analytical method, and the process ends when concentration of formula (V) is between 0-20%, preferably from 0-10%. In particular, the process should be terminated when no more than 1% of compound of formula (IV) remains in the reaction media.

In another embodiment, the present invention provides a method for preparing of a compound of formula (VII), and its salt thereof,

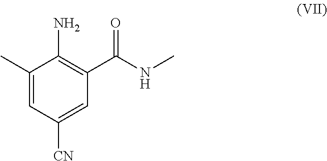

comprising a) reaction of a compound of formula (IV) or its salt thereof, prepared according to the present invention by dehydration of a compound of formula (I), optionally in the presence of a solvent, with thionyl chloride, sulfuryl chloride, phthaloyl chloride, phosphorus pentachloride, phosphorus trichloride, cyanuric chloride, acetic anhydride, propionic anhydride, butyric anhydride, hexanoic anhydride, benzoic anhydride, trichloroacetic anhydride, isopropenyl acetate, acetyl chloride, propionyl chloride, isobutyryl chloride, benzoyl chloride, dicyclohexylcarbodiimide (DCC), diisopropylcarbodiimide (DIC), ethyl-(N′,N′-dimethylamino) propylcarbodiimide hydrochloride (EDC), 1-hydroxybenzotriazole (HOBt), and a mixture thereof, optionally in the presence of a base to form a compound of formula (VI)

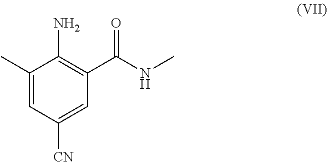

wherein R¹, is halide, optionally halogenated C1-C6-carboxylic acid, carbodiimide, hydroxytriazoles;
and b) reacting the compound of formula (VI) with methylamine or its salts thereof, in the presence of a solvent and optionally in the presence of a base.

According to an embodiment the base in steps a) and b) is selected from a group comprising triethylamine, dimethylamine, aniline, indole, piperidine, pyridine, pyrimidine, pyrrolidine, pyrrole, imidazole, methylimidazole, 2-picoline, 4-methylmorpholine, dimethylaminopyridine, N,N-diisopropylethylamine, sodium carbonate, sodium bicarbonate, sodium hydroxide, sodium acetate, potassium carbonate, potassium bicarbonate, potassium hydroxide, aluminum hydroxide, calcium hydroxide, iron hydroxide, lithium hydroxide, ammonium hydroxide, ammonium acetate, and a mixture thereof.

According to an embodiment the base in steps a) and b) is selected from a group comprising triethylamine, imidazole, sodium carbonate, sodium bicarbonate, sodium acetate, potassium carbonate, potassium bicarbonate, and a mixture thereof.

According to an embodiment, a molar ratio between compound (IV) to the base in step a) can be from about 1:20 to 1:0.1, preferably from 1:10 to 1:1, most preferably from about 1:1.5 to 1:1.

According to an embodiment, a molar ratio between compound (IV) to the base in step a) can be from about 1:10 to 1:1, preferably from 1:5 to 1:1, most preferably from about 1:1.3 to 1:1.1.

According to an embodiment, a molar ratio between compound (IV) to the base in step b) can be from about 1:20 to 1:0.1, preferably from 1:10 to 1:1, most preferably from about 1:1.5 to 1:1.

According to an embodiment, a molar ratio between compound (IV) to the base in step b) can be from about 1:10 to 1:1, preferably from 1:5 to 1:1, most preferably from about 1:1.3 to 1:1.1

According to an embodiment the solvent in steps a) and b) is selected from a group comprising aliphatic cyclic and acyclic hydrocarbons, such as octane, heptane, hexane, pentane, cyclooctane, cyclohexane, cyclopentane petroleum ether, halogenated aliphatic cyclic and acyclic hydrocarbons such as, carbontetrachloride, chloroform, methylenechloride, 1,2-dichloroethane, aromatic hydrocarbons such as benzene, toluene, xylene, ethylbenzene, halogenated aromatic hydrocarbons such as chlorobenzene, dichlorobenzene, trichlorobenzene, aliphatic and cyclic ethers such as diethyl ether, diglyme (diethylene glycol dimethyl ether), 1,4-dioxane, methyl t-butyl ether (MTBE), isopropylmethyl ether, tetrahydrofuran (THF), methyl-tetrahydrofuran (Me-THF), cyclopentylmethyl ether, aliphatic esters such as ethyl acetate nitriles such as acetonitrile, benzonitrile, ketones such as acetone, 2-butanone, C1-C6 alcohols such as methanol, ethanol, 1-butanol, 2-butanol, 1-propanol, 2-propanol, t-butyl alcohol, diethylene glycol, glycerin, ethylene glycol, propylene glycol polar protic and aprotic solvents such as N-methyl-2-pyrrolidone, dimethylformamide, dimethylacetamide, dimethylsulfoxide, water, and a mixture thereof.

According to an embodiment the solvent in steps a) and b) is selected from a group comprising halogenated aliphatic hydrocarbons such as, carbontetrachloride, aliphatic and cyclic ethers such as 1,4-dioxane, methyl t-butyl ether (MTBE), isopropylmethyl ether, tetrahydrofuran (THF), methyl-tetrahydrofuran (Me-THF), nitriles such as acetonitrile, ketones such as acetone, 2-butanone, C1-C6 alcohols such as methanol, ethanol, 1-butanol, 2-propanol, polar protic and aprotic solvents such as N-methyl-2-pyrrolidone, dimethylformamide, dimethylacetamide, dimethylsulfoxide, water, and a mixture thereof.

According to an embodiment, a w/w ratio between the compound of formula (IV) to the solvent in step a) can be from about 1:20 to 10:1, preferably from about 1:0.5 to 1:5, most preferably from about 1:3 to 1:5.

According to an embodiment, a w/w ratio between the compound of formula (IV) to the solvent in step a) can be from about 1:10 to 1:1, preferably from about 1:5 to 1:1, most preferably from about 1:3 to 1:4.

According to an embodiment, a w/w ratio between the compound of formula (VI) to the solvent in step b) can be from about 1:20 to 10:1, preferably from about 1:0.5 to 1:5, most preferably from about 1:3 to 1:5.

According to an embodiment, a w/w ratio between the compound of formula (VI) to the solvent in step b) can be from about 1:10 to 1:1, preferably from about 1:5 to 1:1, most preferably from about 1:3 to 1:4.

According to an embodiment, compound of formula (IV) contacted with any of thionyl chloride, sulfuryl chloride, phthaloyl chloride, phosphorus pentachloride, phosphorus trichloride, cyanuric chloride, acetic anhydride, propionic anhydride, butyric anhydride, hexanoic anhydride, benzoic anhydride, trichloroacetic anhydride, isopropenyl acetate, acetyl chloride, propionyl chloride, isobutyryl chloride, benzoyl chloride and a mixture thereof, is performed between the temperature interval of from about 0 to 120° C. A preferred temperature interval is from about 70 to 120° C., more preferably from about 70 to 100° C.

According to an embodiment, a w/w ratio between the compound of formula (VI) to any of thionyl chloride, sulfuryl chloride, phthaloyl chloride, phosphorus pentachloride, phosphorus trichloride, cyanuric chloride, acetic anhydride, propionic anhydride, butyric anhydride, hexanoic anhydride, benzoic anhydride, trichloroacetic anhydride, isopropenyl acetate, acetyl chloride, propionyl chloride, isobutyryl chloride, benzoyl chloride and a mixture thereof, can be from about 1:1 to 1:10, preferably from about 1:1 to 1:5, most preferably from about 1:1.5 to 1:4.

According to an embodiment, compound of formula (IV) contacted with any of dicyclohexylcarbodiimide (DCC), diisopropylcarbodiimide (DIC), 1-hydroxybenzotriazole (HOBt) and a mixture thereof, is performed between the temperature interval of from about 0 to 120° C. A preferred temperature interval is from about 0 to 50° C., more preferably from about 0 to 30° C.

According to an embodiment, a w/w ratio between the compound of formula (VI) to any of dicyclohexylcarbodiimide (DCC), diisopropylcarbodiimide (DIC), 1-hydroxybenzotriazole (HOBt) and a mixture thereof, can be from about 1:1 to 1:10, preferably from about 1:1 to 1:3, most preferably from about 1:1.1 to 1:1.5.

According to an embodiment, the compound of formula (VI) contacted with methylamine, or its salt thereof is performed between the temperature interval of from about 0 to 120° C., more preferably from about 10 to 70° C.

According to an embodiment, the compound of formula (VI) contacted with methylamine, or its salt thereof is performed between the temperature interval of from about 0 to 60° C.

According to an embodiment the reaction mixture of step a) is monitored by HPLC analytical method, and the process ends when concentration of formula (IV) is between 0-99%, preferably from 0-50%. In particular, the process should be terminated when no more than 1% of compound of formula (IV) remains in the reaction media.

According to an embodiment the reaction mixture of step a) is monitored by HPLC analytical method, and the process ends when concentration of formula (IV) is between 0-20%, preferably from 0-10%. In particular, the process should be terminated when no more than 1% of compound of formula (IV) remains in the reaction media.

According to an embodiment the reaction mixture of step b) is monitored by HPLC analytical method, and the process ends when concentration of formula (VI) is between 0-99%, preferably from 0-50%. In particular, the process should be terminated when no more than 1% of compound of formula (VI) remains in the reaction media.

According to an embodiment the reaction mixture of step b) is monitored by HPLC analytical method, and the process ends when concentration of formula (VI) is between 0-20%, preferably from 0-10%. In particular, the process should be terminated when no more than 1% of compound of formula (VI) remains in the reaction media.

In another aspect of this invention, the compound of formula (I) can be used as an intermediate for preparation of cyantraniliprole. As non-limiting example of preparation of cyantraniliprole, the compound of formula (I) can be at first transformed to intermediates of formula (IV) and/or (VII) as disclosed in the present invention, and then reacted with the corresponding pyrazole carboxylic acid or its derivates by the methods such as but not limited to those disclosed in WO2006068669, WO 2004/067528, WO 2006/062978.

In another aspect of this invention, the compound of formula (II), is prepared according to the present invention can be used as an intermediate for preparation of cyantraniliprole. For the preparation of cyantraniliprole, the compound of formula (II) can be at first transformed to intermediates of formula (IV) and/or (VII), as disclosed in the present invention, and then reacted with the corresponding pyrazole carboxylic acid or its derivates by the methods such as but not limited to those disclosed in WO2006068669, WO 2004/067528, WO 2006/062978.

In another embodiment, the compounds of formula (II), (IV) and (VII) are prepared according to the present invention can be used for preparation of cyantraniliprole using different methods such as, but not limited to those disclosed in WO2006068669, WO 2004/067528, WO 2006/062978.

Without further elaboration, it is believed that one skilled in the art using the preceding description is able to utilize the present invention to its fullest extent. The following Examples are, therefore, to be construed as merely illustrative, and not limiting of the disclosure in any way whatsoever.

### EXPERIMENTAL PART

### Example 1: Preparation of 2-amino-5-formyl-3-methylbenzoic acid from 2-amino-3-methylbenzoic acid

To a 1 L reactor, 2-amino-3-methylbenzoic acid (15.1 gr), acetic acid (30 mL), and methane-sulfonic acid (28 mL) were charged at 25° C. and stirred for 5 minutes. The reaction was then heated to 50° C. and Hexamethylenetetramine (HMTA) (30.8 gr) was added portion wise. The reaction was further heated to 90° C. and the reaction mixture was allowed to stir for 3.5 hours. The reaction progress was monitored by HPLC and ended when the starting material was less than 1% in the reaction mixture. The reaction mixture was cool to 25° C. and hydrochloric acid 32% (54 mL) was added followed by water (500 mL) and the mixture was allowed to stir for 2 hours. 40% aqueous solution of sodium hydroxide (97.5 gr) was added to obtain pH of 5-6, followed by water (100 mL). Precipitant was filtered and washed twice with water (100 mL). The filtered precipitant was transferred to water (300 mL) and the mixture was heated to 60° C. for 1 hour. The mixture was cooled to 30° C. and the precipitant was filtered. The solid precipitant was washed twice with water (50 mL) and transfers to acetonitrile (40 mL). The mixture was allowed to stir for 0.5 hour and the solid was filtered and dried to obtain 11.8 gr of solid product with purity of 83% (55% yield).

### Example 2: Preparation of 2-amino-5-formyl-3-methylbenzoic acid from 2-amino-3-methylbenzoic acid

To a 1 L reactor, 2-amino-3-methylbenzoic acid (40 gr), Hexamethylenetetramine (HMTA) (80 gr) and acetic acid (120 mL) were mixed at 25° C. for 10 minutes. Then, methane-sulfonic acid (76 mL) was added dropwise while maintaining the temperature at 50° C. The reaction was further heated to 85° C. and the reaction mixture was allowed to stir for 2 hours. The reaction progress was monitored by HPLC and ended when the starting material was less than 1% in the reaction mixture. To the hot reaction mixture, water (500 mL) was added, and the mixture was allowed to stir for 20 minutes at 80° C. Then, to this solution, 40% aqueous solution of sodium hydroxide (170 gr) was added to obtain pH of 5, and the mixture was stirred for 12 hours at 30° C. The solid precipitant was filtered and washed with water and the product was dried to obtain 42.6 gr of solid product with purity of 92% (83% yield).

### Example 3 (Comparative): Preparation of 2-amino-5-formyl-3-methylbenzoic acid from 2-amino-3-methylbenzoic acid

To a 1 L reactor, 2-amino-3-methylbenzoic acid (10 gr), acetic acid (18.3 mL), paraformaldehyde (3.27 gr), and Hexamethylenetetramine (HMTA) (10.19 gr) were charged at 25° C. and stirred for 5 minutes. The reaction was then heated to 100° C. and the reaction mixture was allowed to stir for 2 hours. The reaction progress was monitored by HPLC and ended when the starting material was less than 1% in the reaction mixture. To the reaction mixture, hot water of 60° C. (75 mL) was added followed by hydrochloric acid (58 mL) and the mixture was allowed to stir for another 0.5 hours at 90° C. The mixture was cooled to 25° C., and 1M aqueous solution of sodium hydroxide was added to obtain pH=4. After 24 h hours precipitant was filtered and washed twice with water (100 mL). The filtered precipitant was dissolved in hot acetonitrile at 60° C. (50 mL). Acetonitrile was evaporated and the solid was dried to obtain 3.4 gr of yellowish solid product (29% yield).

As shown in Comparative Example 3, the reaction performed in the absence of additional acid (e.g., methane sulfonic acid), the yield decreased significantly.

### Example 4: Preparation of 2-amino-5-((hydroxyimino)methyl)-3-methylbenzoic acid from 2-amino-5-formyl-3-methylbenzoic acid

To a 0.1 L reactor, 2-amino-5-formyl-3-methylbenzoic acid (2.0 gr), hydroxylamine hydrochloride (0.85 gr), and acetonitrile (20 mL) were charged at 25° C. and stirred for 5 minutes. The reaction was then heated to 80° C. and the mixture was allowed to stir for 4 hours. The reaction progress was monitored by HPLC and ended when the starting material was less than 1% in the reaction mixture. The mixture was cooled to 40° C. and water (100 mL) was added dropwise. The mixture was cooled to 10° C. and the participant was filtered. The solid filtrate was washed three times with water (50 mL) and dried to obtain 1.95 gr of solid product (90% yield).

### Example 5: Preparation of 2-amino-5-cyano-3-methylbenzoic acid from 2-amino-5-formyl-3-methylbenzoic acid

To a 0.1 L reactor, 2-amino-5-formyl-3-methylbenzoic acid (2.0 gr), hydroxylamine hydrochloride (0.85 gr), and dimethylsulfoxide (10 mL) were charged at 25° C. and stirred for 5 minutes. The reaction was then heated to 90° C. and the mixture was allowed to stir for 4 hours. The reaction progress was monitored by HPLC and ended when the starting material was less than 1% in the reaction mixture. The mixture was cooled to 40° C. and water (100 mL) was added dropwise. The mixture was cooled to 10° C. and the participant was filtered. The solid filtrate was washed three times with water (50 mL) and dried to obtain 1.55 gr of solid product (63% yield).

### Example 6: Preparation of 8-methyl-2,4-dioxo-1,4-dihydro-2H-benzo[d][1,3]oxazine-6-carbonitrile from 2-amino-5-cyano-3-methylbenzoic acid

To a 0.1 L reactor, 2-amino-5-cyano-3-methylbenzoic acid (5.0 gr), toluene (50 ml), tetrabutylammonium bromide (0.26 gr) and 30% aqueous solution of sodium hydroxide (10.1 mL) was charged at 25° C. and stirred for 5 minutes. To the mixture, triphosgene (3.7 gr) in toluene (24 gr) was added portion-wise maintaining the temperature at 25° C. and the mixture was allowed to stir for 1.5 hours. The reaction progress was monitored by HPLC and ended when the starting material was less than 1% in the reaction mixture. The precipitant was filtered and washed twice with water and dried to obtain 6.0 gr of solid product (91% yield).

### Example 7: Preparation of 8-methyl-2,4-dioxo-1,4-dihydro-2H-benzo[d][1,3]oxazine-6-carbonitrile from 2-amino-5-((hydroxyimino)methyl)-3-methylbenzoic acid

To a 0.1 L reactor, 2-amino-5-((hydroxyimino)methyl)-3-methylbenzoic acid (8.0 gr), triphosgene (24.0 gr), and THF (80.0 mL) were charged at 25° C. and stirred for 5 minutes. The reaction was then heated to 66° C. and the mixture was allowed to stir for 2 hours. The reaction progress was monitored by HPLC and ended when the starting material was less than 1% in the reaction mixture. The reaction mixture was then concentrated to obtain 7.1 gr of the desired product (Yield: 85%).

### Example 8: Preparation of 8-methyl-2,4-dioxo-1,4-dihydro-2H-benzo[d][1,3]oxazine-6-carbonitrile from 2-amino-5-((hydroxyimino)methyl)-3-methylbenzoic acid

To a 0.1 L reactor, 2-amino-5-((hydroxyimino)methyl)-3-methylbenzoic acid (1.0 gr), triphosgene (3.0 gr), and Me-THF (10.0 mL) were charged at 25° C. and stirred for 5 minutes. The reaction was allowed to stir for 2 hours at 25° C. The reaction progress was monitored by HPLC and ended when the starting material was less than 1% in the reaction mixture. The reaction mixture was then concentrated to obtain 0.8 gr of the desired product (Yield: 80%).

### Example 9: Preparation of 2-amino-5-cyano-N,3-dimethylbenzamide from 8-methyl-2,4-dioxo-1,4-dihydro-2H-benzo[d][1,3]oxazine-6-carbonitrile

To a 0.1 L reactor, 8-methyl-2,4-dioxo-1,4-dihydro-2H-benzo[d][1,3]oxazine-6-carbonitrile (2.0 gr), 2-propanol (20 mL), and sodium bicarbonate (2.83 gr) were charged at 25° C. and stirred for 5 minutes. To the mixture, methylammonium chloride (1.7 gr) was added portion-wise maintaining the temperature at 0° C. and the mixture was allowed to stir for 2.5 hours. The reaction progress was monitored by HPLC and ended when the starting material was less than 1% in the reaction mixture. The crude product was filtered off, washed in water and dried to give 1.5 g product (yield: 80%).

### Example 10: Preparation of cyantraniliprole from 2-amino-5-cyano-N,3-dimethylbenzamide

To a 5 L reactor, toluene (2200 gr) and 2-amino-5-cyano-N,3-dimethylbenzamide (215 gr) were charged at 25° C. and stirred for 5 minutes. The mixture was heated to 90° C. and vacuum was applied at 380 mbar. Then, 3-bromo-5-(3-chloropyridin-2-yl)cyclopenta-1,3-diene-1-carbonyl chloride (350 gr) in toluene (1140 gr) was added dropwise while extracting hydrogen chloride and toluene from the reaction. At the end of the addition, reaction mixture was allowed to stir for additional 3 h while toluene was added to maintain the reaction mixture volume at 800 gr. Then, vacuum was stopped, and the mixture was allowed to stir for additional 1 h while purging with nitrogen. The reaction progress was monitored by HPLC and ended when the starting material was less than 1% in the reaction mixture. The mixture was cooled to 0° C. and solid precipitant was filtered. The solid was washed with toluene and vacuum dried at 70° C. The solid was then dissolved in methanol (1600 gr) and mixture was heated to 65° C. for 4 h. Mixture was cooled to 5° C. for 2 h and the precipitant solid was filtered and washed with fresh methanol. The solid was dried under vacuum at 70° C. for 2 hours to obtain 470 gr product (91% yield).

1. A process for preparing of a compound of formula (I), or its salts thereof 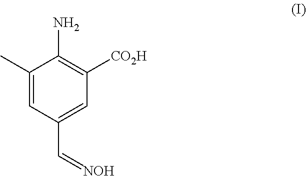
comprising reaction of a compound of formula (II) or its salts thereof 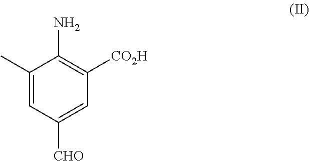
with hydroxylamine, or its salts thereof, optionally in the presence of a solvent, optionally in the presence of a base.

2. The process according to claim 1, wherein the base is selected from a group comprising triethylamine, dimethylamine, pyridine, 2-picoline, 4-methylmorpholine, dimethyl aminopyridine, N,N-diisopropylethylamine, sodium carbonate, sodium bicarbonate, sodium hydroxide, sodium acetate, potassium carbonate, potassium bicarbonate, potassium hydroxide, ammonium hydroxide, ammonium acetate, and a mixture thereof.

3. The process according to claim 1, wherein the solvent is selected from a group comprising aliphatic cyclic and acyclic hydrocarbons, halogenated aliphatic cyclic and acyclic hydrocarbons, aromatic hydrocarbons, halogenated aromatic hydrocarbons, aliphatic and cyclic ethers, aliphatic esters, nitriles, ketones, C1-C6 alcohols, C1-C4 carboxylic acid, formic acid, pyridine, ethylene glycol, propylene glycol, n-alkylpyrrolidones, dimethylformamide, dimethylacetamide, dimethylsulfoxide, water, and a mixture thereof.

4. A process of preparing of a compound of formula (IV), and its salt thereof, 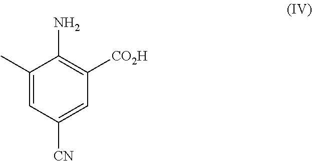
comprising dehydration of a compound of formula (I), optionally in the presence of a solvent.

5. The process according to claim 4, wherein the solvent is selected from a group comprising aliphatic cyclic and acyclic hydrocarbons, halogenated aliphatic cyclic and acyclic hydrocarbons, aromatic hydrocarbons, halogenated aromatic hydrocarbons, aliphatic and cyclic ethers, aliphatic esters, nitriles, ketones, C1-C6 alcohols, C1-C4 carboxylic acids, acetonitrile, pyridine, ethylene glycol, propylene glycol, n-alkylpyrrolidones, dimethylformamide, dimethylacetamide, dimethyl sulfoxide, water, and the mixtures thereof.

6. A process of preparing of a compound of formula (II), and its salt thereof, 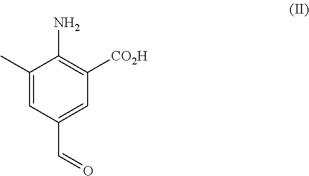
comprising reaction of compound of formula (III), and its salt thereof, 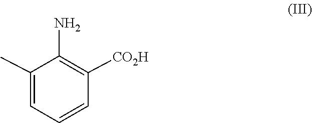
with a formylation reagent selected from hexamethylenetetramine (HMTA), formaldehyde, paraformaldehyde, trioxane and/or methanediol and the mixtures thereof, in the presence of an acid, optionally in the presence of a solvent.

7. The process according to claim 6, wherein the acid is selected from the group comprising, formic acid, triflic acid, monochloroacetic acid, dichloroacetic acid, trichloroacetic acid, trifluoroacetic acid, sulfuric acid, phosphoric acid, nitric acid, methanesulfonic acid, para-toluenesulfonic acid, hydrochloric acid, hydrobromic acid, boric acid, and the mixture thereof.

8. The process according to claim 6, wherein the solvent is selected from a group comprising aliphatic cyclic and acyclic hydrocarbons, halogenated aliphatic cyclic and acyclic hydrocarbons, aromatic hydrocarbons, halogenated aromatic hydrocarbons, aliphatic and cyclic ethers, aliphatic esters, nitriles, ketones, C1-C6 alcohols, n-alkyls protic and aprotic polar solvents such as, pyridine, ethylene glycol, propylene glycol, n-alkylpyrrolidones, dimethylformamide, dimethylacetamide, dimethylsulfoxide, water and the mixtures thereof.

9. A process of preparing of a compound of formula (V), and its salt thereof, 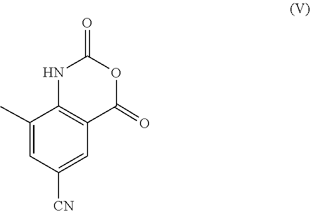
comprising a) reaction of a compound of formula (I) or its salt thereof, prepared according to claim 1, with a compound of formula (A) 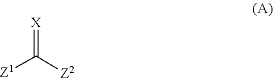
wherein Z¹ and Z², are independently, chloride, C1-C4-alkoxy, trichloromethyloxy, C(O)Cl, C1-C6-alkyloxycarbonate;
X is O;
in the presence of a solvent. optionally in the presence of a base, optionally in the presence of a phase transfer catalyst

10. The process according to claim 9, wherein the solvent is selected from a group comprising aliphatic cyclic and acyclic hydrocarbons, halogenated aliphatic cyclic and acyclic hydrocarbons, aromatic hydrocarbons, halogenated aromatic hydrocarbons, aliphatic and cyclic ethers, aliphatic esters, nitriles, ketones, C1-C6 alcohols, n-alkyls protic and aprotic polar solvents such as, pyridine, ethylene glycol, propylene glycol, n-alkylpyrrolidones, dimethylformamide, dimethylacetamide, dimethylsulfoxide, water and the mixtures thereof.

11. A process of preparing of a compound of formula (VII), and its salt thereof, 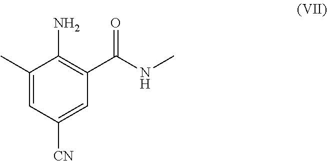
comprising a) reaction of a compound of formula (I) or its salt thereof, prepared according to claim 1, with a compound of formula (A) 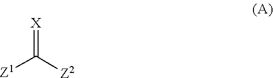
wherein Z¹ and Z², are independently, chloride, C1-C4-alkoxy, trichloromethyloxy, C(O)Cl, C1-C6-alkyloxycarbonate;
X is O;
in the presence of a solvent
and b) further reacting compound of formula (V) with methylamine or its salts thereof, in the presence of a solvent and optionally in the presence of a base.

12. The process according to claim 11 wherein the solvent is selected from a group comprising aliphatic cyclic and acyclic hydrocarbons, halogenated aliphatic cyclic and acyclic hydrocarbons, aromatic hydrocarbons, halogenated aromatic hydrocarbons, aliphatic and cyclic ethers, aliphatic esters, nitriles, ketones, dimethyl carbonate, dimethylformamide, pyridine, dimethylsulfoxide, n-alkylpyrrolidones, C1-C6-alkoxy, C1-C4-carboxylic acid, water, and a mixture thereof.

13. The process according to claim 11, wherein the base is selected from a group comprising triethylamine, pyridine, 2-picoline, 4-methylmorpholine, dimethyl aminopyridine, N,N-diisopropylethylamine, sodium carbonate, sodium bicarbonate, sodium hydroxide, potassium carbonate, potassium bicarbonate, potassium hydroxide, ammonium hydroxide and a mixture thereof.

14. A process of preparing of a compound of formula (VII), and its salt thereof, 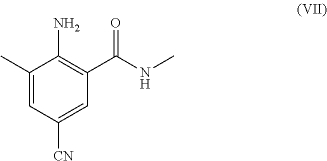
comprising a) reaction of a compound of formula (IV) or its salt thereof, prepared according to claim 4, with a compound of formula (A) 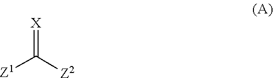
wherein Z¹ and Z², are independently, chloride, C1-C4-alkoxy, trichloromethyloxy, C(O)Cl, C1-C6-alkyloxycarbonate;
X is O;
in the presence of a solvent, optionally in the presence of a base, optionally in the presence of a phase transfer catalyst to obtain a compound of formula (V), 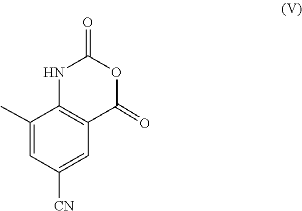
and b) reacting the compound of formula (V) with methylamine or its salts thereof, in the presence of a solvent and optionally in the presence of a base.

15. The process according to claim 14, wherein the base is selected from a group comprising triethylamine, pyridine, 2-picoline, 4-methylmorpholine, dimethyl aminopyridine, N,N-diisopropylethylamine, sodium carbonate, sodium bicarbonate, sodium hydroxide, potassium carbonate, potassium bicarbonate, potassium hydroxide, ammonium hydroxide and a mixture thereof.

16. The process according to claim 14 performed in the presence of phase transfer catalyst selected from quaternary ammonium salts, phosphonium salts, crown ethers, polyether's and the mixtures thereof.

17. The process according to claim 14, wherein the phase transfer catalyst selected from a group comprising benzyltriethylammonium chloride, methyltricaprylammonium chloride, methyltributylammonium chloride, and methyltrioctylammonium chloride, tetra-n-butylammoniumchloride, tetra-n-butylammonium bromide, tetra-n-butylammonium iodide, tetra-n-butylammonium fluoride, crown ethers, polyethylene glycols, polypropylene glycols, and the mixture thereof.

18. The process according to claim 14 wherein the solvent in step a) is selected from a group comprising aliphatic cyclic and acyclic hydrocarbons, halogenated aliphatic cyclic and acyclic hydrocarbons, aromatic hydrocarbons, halogenated aromatic hydrocarbons, aliphatic and cyclic ethers, aliphatic esters, nitriles, ketones, dimethyl carbonate, dimethylformamide, pyridine, dimethylsulfoxide, n-alkylpyrrolidones, and a mixture thereof.

19. The process according to claim 14 wherein the solvent in step b) is selected from a group comprising aliphatic cyclic and acyclic hydrocarbons, halogenated aliphatic cyclic and acyclic hydrocarbons, aromatic hydrocarbons, halogenated aromatic hydrocarbons, aliphatic and cyclic ethers, aliphatic esters, nitriles, ketones, dimethyl carbonate, dimethylformamide, pyridine, dimethylsulfoxide, n-alkylpyrrolidones, C1-C6-alkoxy, C1-C4-carboxylic acid, water, and a mixture thereof.

20. A process of preparing of a compound of formula (VII), and its salt thereof, 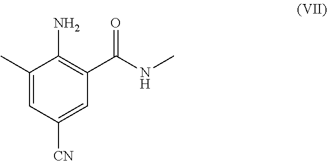
comprising a) reaction of a compound of formula (IV) or its salt thereof, prepared according to claim 4, with thionyl chloride, sulfuryl chloride, phthaloyl chloride, phosphorus pentachloride, phosphorus trichloride, cyanuric chloride, acetic anhydride, propionic anhydride, butyric anhydride, hexanoic anhydride, benzoic anhydride, trichloroacetic anhydride, isopropenyl acetate, acetyl chloride, propionyl chloride, isobutyryl chloride, benzoyl chloride, dicyclohexylcarbodiimide (DCC), diisopropylcarbodiimide (DIC), ethyl-(N′,N′-dimethylamino) propylcarbodiimide hydrochloride (EDC), 1-hydroxybenzotriazole (HOBt), and a mixture thereof, optionally in the presence of a base to form a compound of formula (VI) 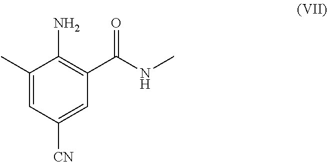
wherein R¹, is halide, optionally halogenated C1-C6-carboxylic acid, carbodiimide, hydroxytriazoles;
and b) reacting the compound of formula (VI) with methylamine or its salts thereof, in the presence of a solvent and optionally in the presence of a base.

21. The process according to claim 20, wherein the solvent is selected from a group comprising cyclic and acyclic aliphatic carbohydrates, halogenated aliphatic cyclic and acyclic hydrocarbons, aromatic hydrocarbons, halogenated aromatic hydrocarbons, aliphatic and cyclic ethers, aliphatic, nitriles, ketones, C1-C6 alcohols, n-alkylpyrrolidones, dimethylformamide, pyridine, dimethylsulfoxide, 1,2-dimethoxyethane, ethylene glycol, water, and a mixture thereof.

22. The process according to claim 20, wherein the base is selected from a group comprising triethylamine, pyridine, 2-picoline, 4-methylmorpholine, dimethyl aminopyridine, N,N-diisopropylethylamine, sodium carbonate, sodium bicarbonate, sodium hydroxide, potassium carbonate, potassium bicarbonate, potassium hydroxide, ammonium hydroxide and a mixture thereof.

23. A process of preparing of a compound of formula (VIII), and its salt thereof, 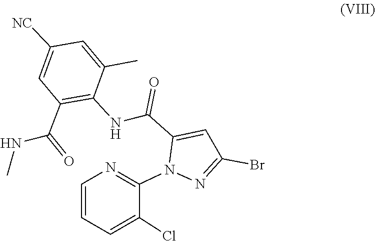 [SMILES: CNC(=O)c1cc(C#N)cc(C)c1NC(=O)c1cc(Br)nn1-c1ncccc1Cl]
using a compound of formula (I).

24. A process of preparing of a compound of formula (VIII), and its salt thereof, 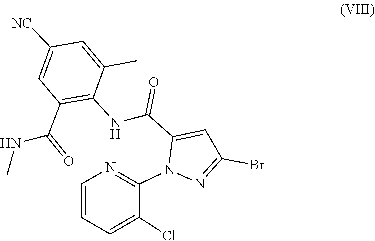 [SMILES: CNC(=O)c1cc(C#N)cc(C)c1NC(=O)c1cc(Br)nn1-c1ncccc1Cl]
using a compound of formula (II), prepared according to claims 6-8.

25. A process of preparing of a compound of formula (VIII), and its salt thereof, 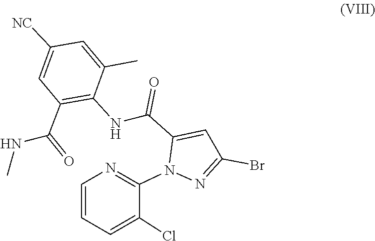 [SMILES: CNC(=O)c1cc(C#N)cc(C)c1NC(=O)c1cc(Br)nn1-c1ncccc1Cl]
using a compound of formula (IV), prepared according to claims 4-5.

26. A process of preparing of a compound of formula (VIII), and its salt thereof, 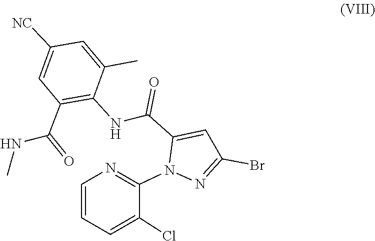 [SMILES: CNC(=O)c1cc(C#N)cc(C)c1NC(=O)c1cc(Br)nn1-c1ncccc1Cl]
using a compound of formula (V), prepared according to any of claims 9-10.

27. A process of preparing of a compound of formula (VIII), and its salt thereof, 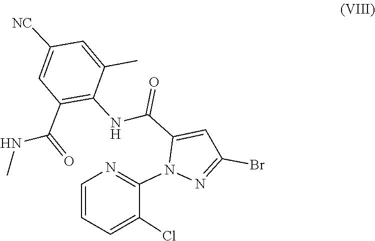 [SMILES: CNC(=O)c1cc(C#N)cc(C)c1NC(=O)c1cc(Br)nn1-c1ncccc1Cl]
using a compound of formula (VII), prepared according to any of claims 11-13, or 14-19 or 20-22.
